(19) United States
(12) Patent Application Publication
(10) Pub. No.: US 2007/0046895 A1
(43) Pub. Date: Mar. 1, 2007

(54) Eye exercise device and method

(76) Inventor: Maxim D. Levinrad, Boca Raton, FL (US)

Correspondence Address: CHRISTIE, PARKER & HALE, LLP, PO BOX 7068, PASADENA, CA, 91109-7068, US

(21) Appl. No.: 11/510,050

(22) Filed: Aug. 24, 2006

Related U.S. Application Data
(60) Provisional application No. 60/711,133, filed on Aug. 24, 2005.

(51) Int. Cl. A61B 3/00

(52) U.S. Cl. 351/203

(57) ABSTRACT

An eye exercise device and method for strengthening the eyes. The eye exercise device has a first portion, a second portion, a mirror, and viewing cards. The first portion has a first end and a second end with a longitudinal channel formed therethrough. The second portion has a longitudinal dimension and is positioned at an angle relative to the first portion in a connection region of the first and second portions. The second portion has at least one viewing card holder. A mirror is located in the connection region and directs light through the first and second portions. A selected viewing card is adapted to be retained by the viewing card holder. The viewing card has graphic indicia adapted to exercise the eyes by requiring a user to focus for an interval of time on the graphic indicia to strength the eye muscles.

### CROSS-REFERENCE TO RELATED APPLICATIONS

This application claims the benefit of provisional patent application No. 60/711,133, filed on Aug. 24, 2005.

### SUMMARY OF THE INVENTION

The invention relates to the field of vision stabilization and improvement, and more specifically to an eye exercise device for improving and strengthening the muscles of the eye to stabilize and improve vision, whether myopia (nearsightedness) or presbyopia (farsightedness). It is known that presbyopia has as a main cause the weakening of the muscles on the outside of the eyeballs, usually due to aging. The most common way this is dealt with is to use reading glasses, which magnify the reading material.

It has also been known for many decades that certain eye exercises are available to improve vision, and many articles and books have been written on this subject. Indeed, between 1919 and 1930, the ophthalmologist Dr. William Bates wrote about his breakthroughs in vision improvement in his magazine Better Eyesight. In October 2000, the book “Better Eyesight: The Complete Magazine of William H. Bates” was published.

While certain eye exercises are disclosed in the literature, there are no specific devices designed to help users to improve their vision in a step-by-step manner, and therefore it can be difficult not only to gauge progress while following the eye improvement method, but also can be troublesome.

There accordingly remains a need for a device to exercise the eyes for improved and stabilized vision.

### BRIEF DESCRIPTION OF THE DRAWINGS

FIG. 1 is a right side isometric view of an exemplary embodiment of an eye exercise device of the invention, with its viewing card removed.

FIG. 2 is a right side view of the eye exercise device of FIG. 1, shown without a viewing card in place.

FIG. 3 is a right side view of the eye exercise device of FIG. 1, with a viewing card in an uppermost position so as to establish a first focal distance.

FIG. 4 is a right side view of the eye exercise device of FIG. 1, with a viewing card in a lower position so as to establish a second, longer focal distance.

FIG. 5 is a right side view of the eye exercise device of FIG. 1, with a viewing card in a penultimate lowest position to establish a longer yet focal distance.

FIG. 6 is a right side view of the eye exercise device of FIG. 1, with a viewing card in its lowest position to establish the longest focal distance.

FIG. 7 is a right side view of another embodiment of the eye exercise device of the invention, shown with an illuminating device.

FIG. 8A is a top plane view of a first exemplary viewing card of a set for use with the eye exercise device of FIGS. 1-7, having graphic indicia, namely text in a mirror image, have a first, largest font size.

FIG. 8B-F are top plane views of additional exemplary viewing cards of the set of FIG. 8A for use with the eye exercise device of FIGS. 1-7, having graphic indicia, namely text in mirror image, have a second through sixth, respectively, smaller font sizes than the text on the viewing card of FIG. 8A.

FIG. 9 is a top plane view of another exemplary viewing card for use with the eye exercise device of FIGS. 1-7, having graphic indicia, namely a grid pattern in the form of a “magicube”.

FIG. 10 is a diagrammatic view showing the exemplary embodiment of the eye exercise device in use.

### DETAILED DESCRIPTION OF THE INVENTION

The invention is now described in detail with reference to the figures.

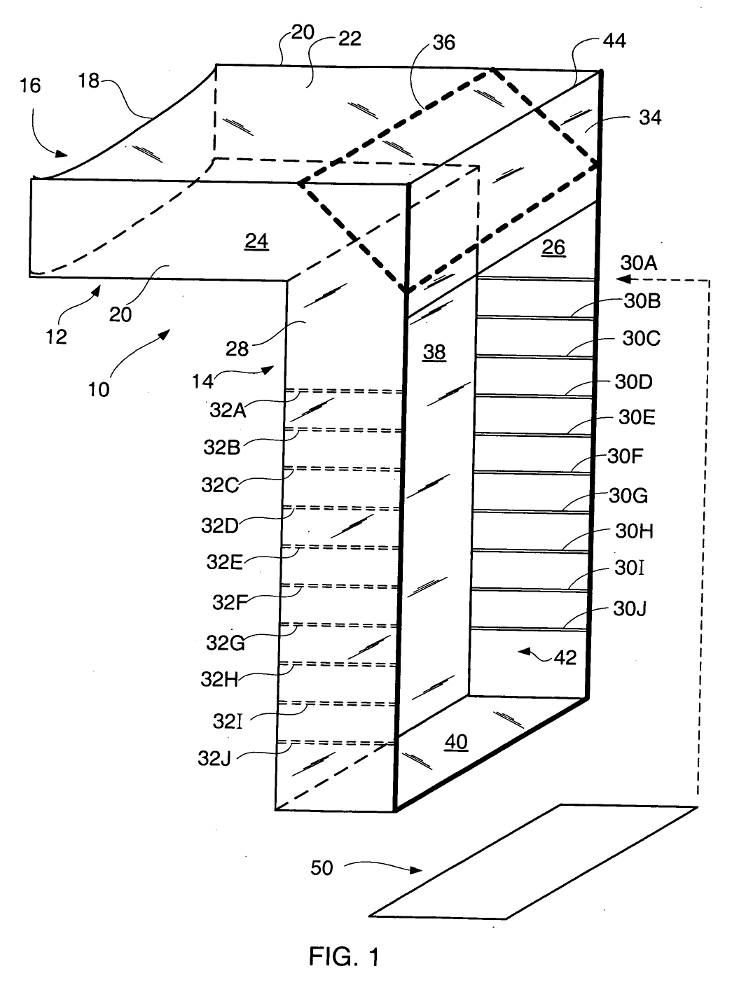

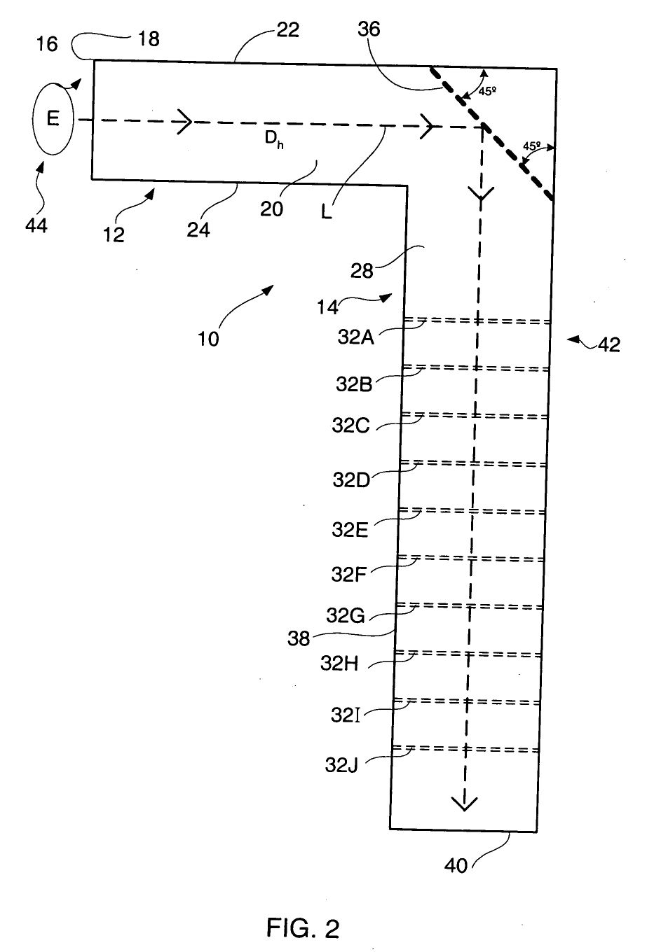

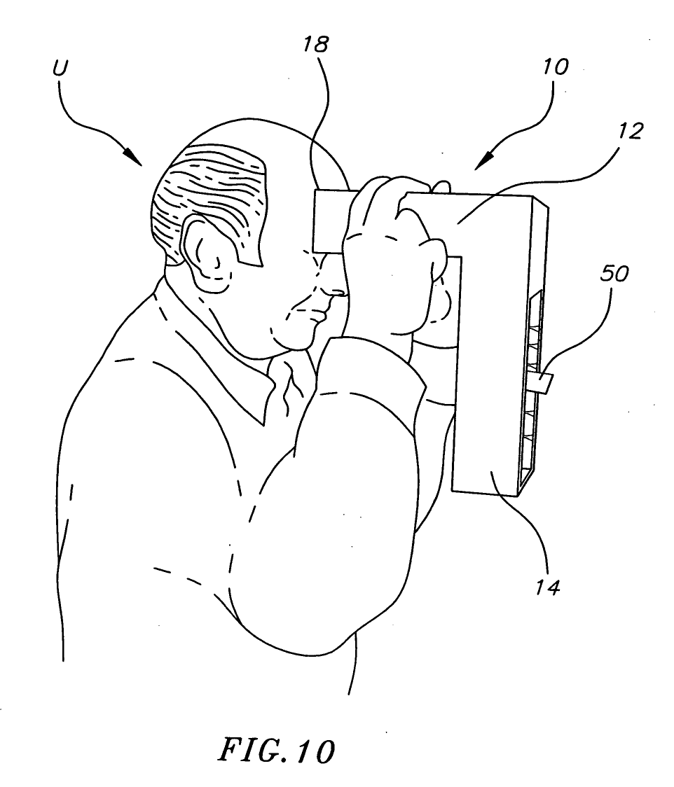

FIG. 1 is a right side isometric view of an exemplary embodiment of the invention, with its viewing card 50 removed. The exemplary embodiment of an eye exercise device 10 has a first portions (hereinafter “horizontal portion”) 12 and a second portion (hereinafter sometimes “vertical portion”) 14. A front 16 of the horizontal portion 12 has a contact surface 18 which can preferably be convexly curved in order to better seat on the user's face around the eyes (as shown in FIG. 10). The horizontal portion 12 has side walls 20, a top wall 22 and a bottom wall 24 and forms a generally enclosed tunnel-like portion with a longitudinal light pathway being formed therethrough. The horizontal portion 12 joins with the vertical portion 14. Vertical portion 14 has side walls 26 and 28 with a series of vision card holders 30A-J and 32A-J, respectively. The vertical portion 14 also has a front wall 38 and a bottom wall 40 and is open at a rear 42 in order to permit light to enter and the viewing card 50 to be positioned at a desired level. The vision card holders 30A-J and 32A-J can comprise, for example, a series of parallel slots formed on the insides of side walls 26 and 28. Other holders can be used such as to allow a series of preset positions, or a continuous adjustment, such as a sliding rail that holds the vision card. The vision card holders 30A-J and 32A-J, which are shown as numbering 10 on each side, can be less or more in number, but will be preferably parallel to each other and slots on the opposite sidewalls, and are located at the same level each (e.g., 30C and 32C) on the opposite sidewalls 26 and 28. A section of material 34 is located at an upper end of the vertical portion 14. A mirror 36 is located in the eye exercise device in a corner 44 (interface region) opposite the open front 16, and is positioned at about 452 from the top wall 22 of the horizontal portion 12 and, also about a 452 angle from the section of material 34, which is best shown in FIGS. 2-7. Thus, light traveling in a direction horizontally through the horizontal portion 12 will be reflected at about a 90° angle and travel vertically through the vertical portion 14 and perpendicular to the vision card holders 30A-J and 32A-J (and a vision card 50 located therein.) If desired, the angle between the horizontal portion 12 and the vertical portion 14 can be changed to something different than a 90° angle, in which case the angle of the mirror would be adjusted to properly reflect the light. In cases where the vision card holders 30A-J and 32A-J comprise slots, the view card 50 will have a width and thickness matched to permit the viewing card to be slid into the slots to locate the viewing card in the eye exercise device 10. Regardless of how the viewing card is retained in position, it will be preferably be perpendicular to the path of light a person will see when using the device 10. The eye exercise device 10 can be made of materials such as plastic, paper (e.g., cardboard or cardstock), wood, metal, glass or combinations thereof, or other materials, and for greater lighting capability, a light source can be included in the device to help illuminate the viewing cards. A back wall, if provided, can be clear or translucent, if desired, to increase the amount of light impinging on the viewing card, and would have openings formed therein to permit placement of the viewing cards. The horizontal portion 12 will preferably be opaque. If desired, the horizontal portion 12 and the vertical portion 14 can be made to hinge relative to each other and brought closer together for compact storage when not in use (not shown) . If desired, the second portion 14 can also be as simple as a single bar or rod that has a single position adjustable device for holding the viewing cards, or a series of fixed position viewing card holders.

FIG. 2 is a right side view of the eye exercise device 10 of FIG. 1, shown without a viewing card in place. The direction of light travel is shown as a dashed line L. By way of example, and not as a limitation, in one embodiment of the eye exercise device 10, the horizontal portion 12 can be approximately 9.5 inches long and the vertical portion 14 can be approximately 16 inches long. Also, by way of example, and not as a limitation, in one embodiment of the eye exercise device, the distance from the front 18 of the horizontal portion 12 to the middle of the mirror 36 Dₕ can be approximately 8 inches, and the distance from the middle of the mirror 36 vertically downwardly will vary depending on what position the vision card (not shown) is located. The vision card holders 30A-J and 32A-J will be spaced apart from each other by a set amount, e.g., in one inch increments. The ability to read written material at about 12 inches from the eyes is considered ideal, and the position of the uppermost vision card holders 30A and 32B are set so that the effective focal length from the open front 16 of the device 10 to the first vision card level will be about 12 inches. Other shorter focal lengths can be used if desired. The terms “horizontal” and “vertical” are for ease of reference and in use, the orientation of these portions can be varied.

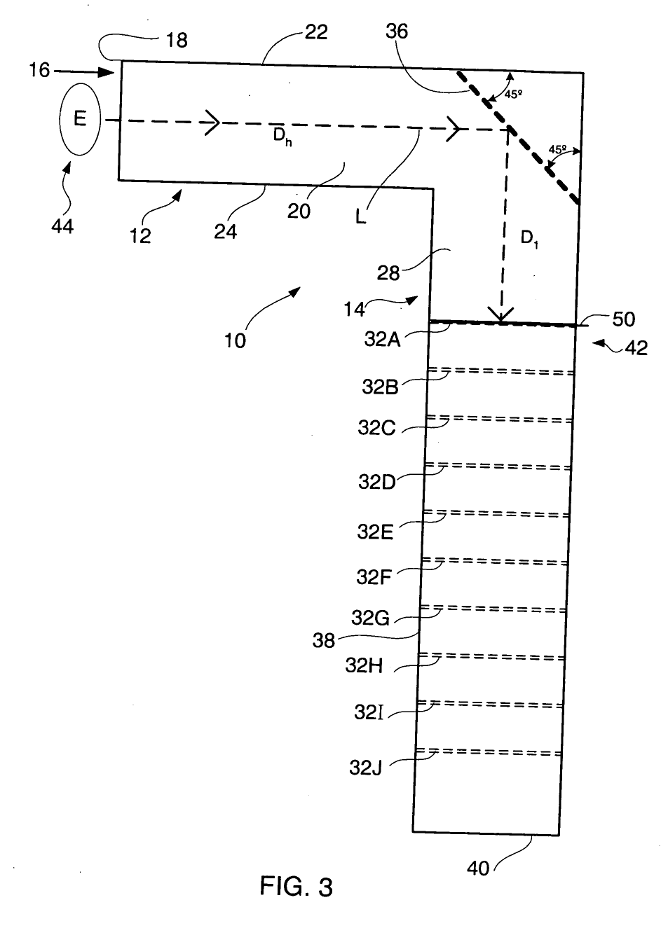

FIG. 3 is a right side view of the eye exercise device 10 of FIG. 1, with a viewing card 50 in an uppermost position defined by vision card holders 32A (30A not shown) so as to establish a first focal distance of 12 inches. The direction of light travel is shown as a dashed line L. For a focal length FL of about 12 inches, the uppermost vision card holders 30A and 32A positions the viewing card 50 at a distance D₁ of about 4 inches from the center of the mirror to produce the desired focal length of 12 inches (Dₕ+D₁=FL₁).

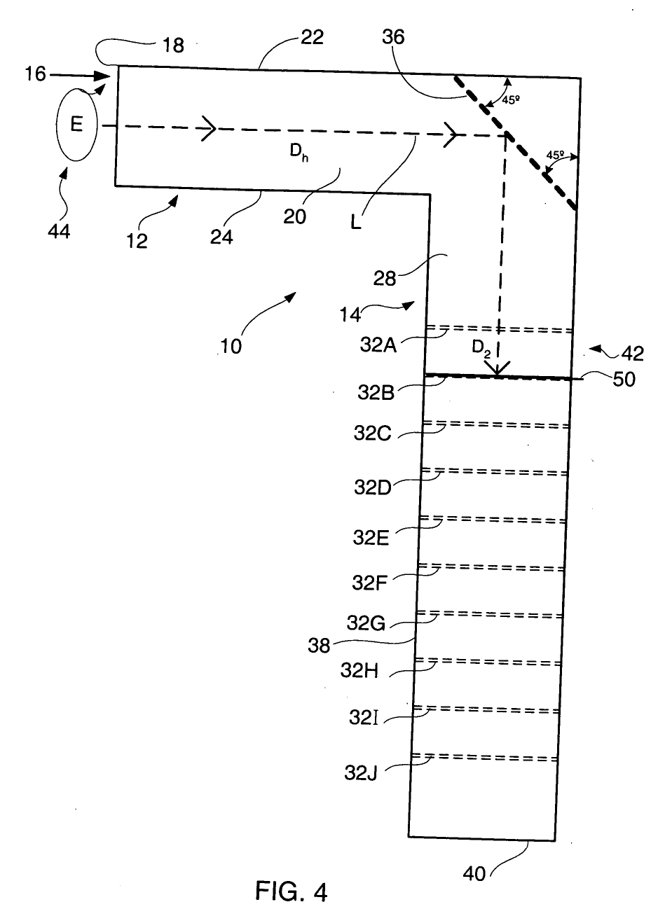

FIG. 4 is a right side view of the eye exercise device of FIG. 1, with a viewing card in a lower position 30B and 32B so as to establish a second, longer focal distance of 13 inches (8 inches+5 inches=13 inches (Dₕ+D₂=FL₂)) .

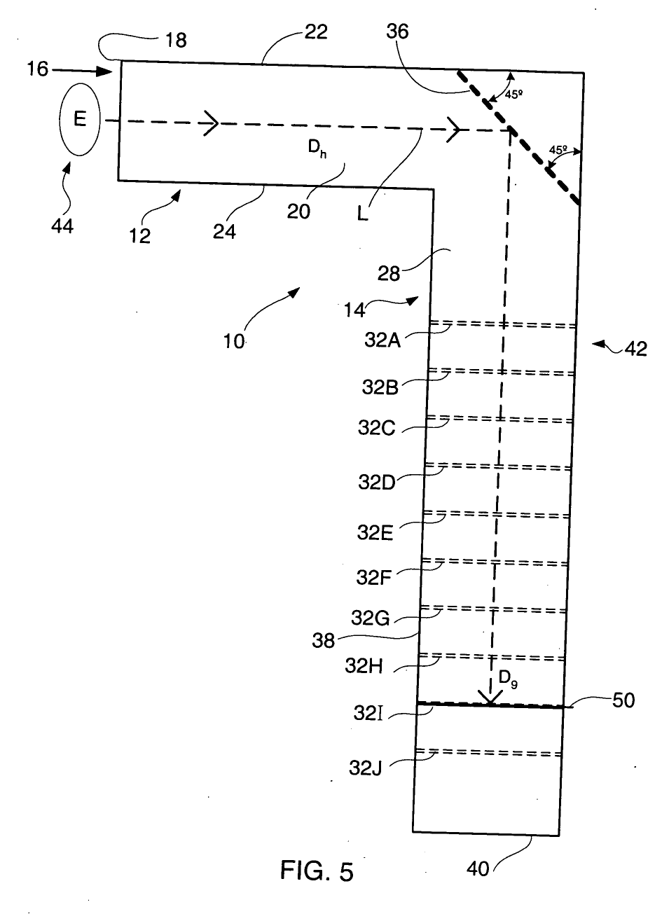

FIG. 5 is a right side view of the eye exercise device of FIG. 1, with a viewing card 50 in a penultimate lowest position so as to establish a ninth, even longer focal distance of 20 inches (8 inches+12 inches=20 inches (Dₕ+D_{g}=FL₉)).

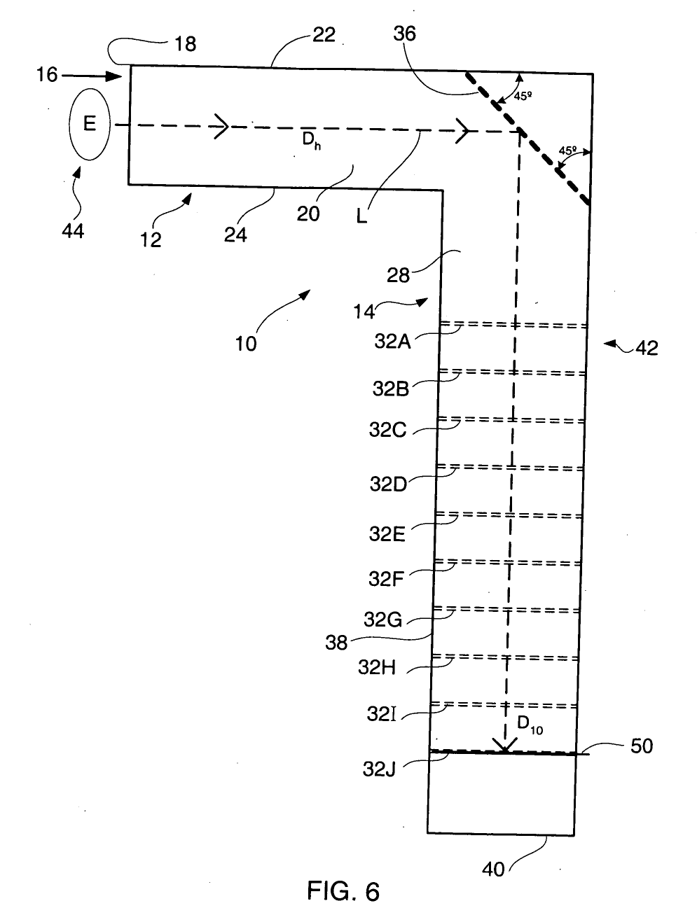

FIG. 6 is a right side view of the eye exercise device of FIG. 1, with a viewing card 50 in a penultimate lowest position so as to establish a lowest and longest focal distance of 21 inches (8 inches+13 inches=21 inches (Dₕ+D₁₀=FL₁₀)).

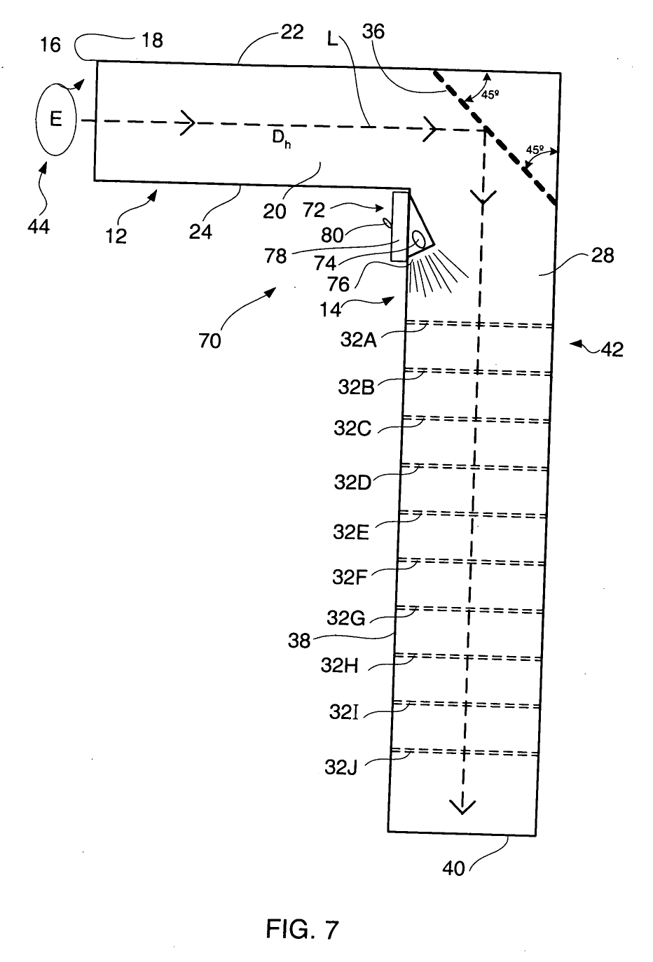

FIG. 7 is a right side view of another embodiment of the eye exercise device 70 of the invention, shown with an exemplary illuminating device 72 for projecting light downwardly into the vertical portion 14 of the eye exercise device 70. The illuminating device 72 has a light source 74, such as an incandescent light bulb, LED, etc. 74 a lower opening 76, an energy source 78 (e.g., a battery) and a switch 80. Other types of illuminating devices can be used with the eye exercise device 70 in order to provide good illumination, even when the ambient light in the location where the device is being used is not sufficient. In this embodiment, the illuminating device 72 can obviate the need for an open back 42 of the vertical portion 14 to allow external light to enter, and therefore the back can be closed up except for slots that would be aligned with the vision card holders 30 and 32.

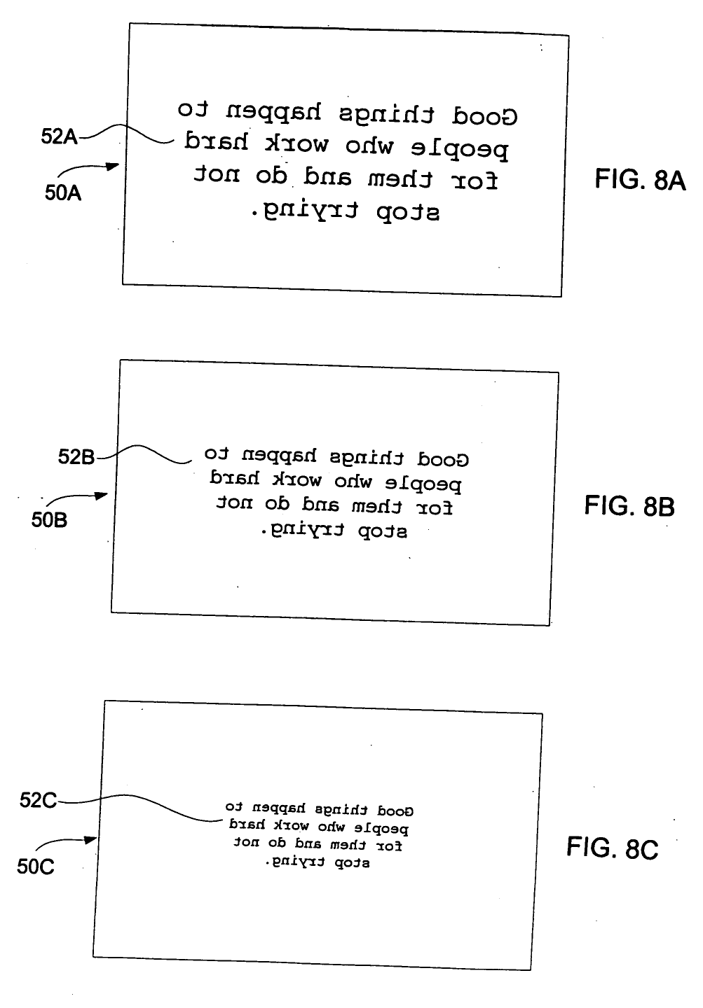

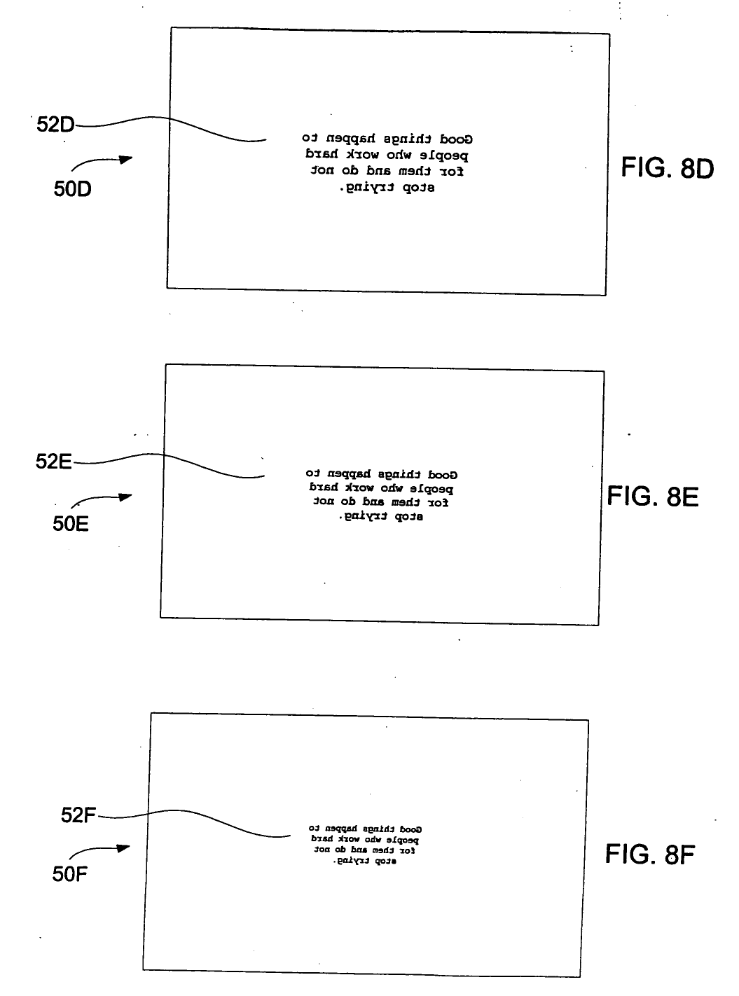

FIG. 8A is a top plane view of a first exemplary viewing card 50A of a set for use with the eye exercise device of FIGS. 1-7, having graphic indicia, namely text 52A, preferably in a mirror image, have a first, largest font size.

FIG. 8B-F are top plane views of additional exemplary viewing cards 50B-F of the set of FIG. 8A for use with the eye exercise device of FIGS. 1-7, having graphic indicia, namely text 52B-F in mirror image, have a second through sixth, smaller font sizes than the text on the viewing card of FIG. 8A. While the viewing card shows mirror image text in English, other languages can obviously be used.

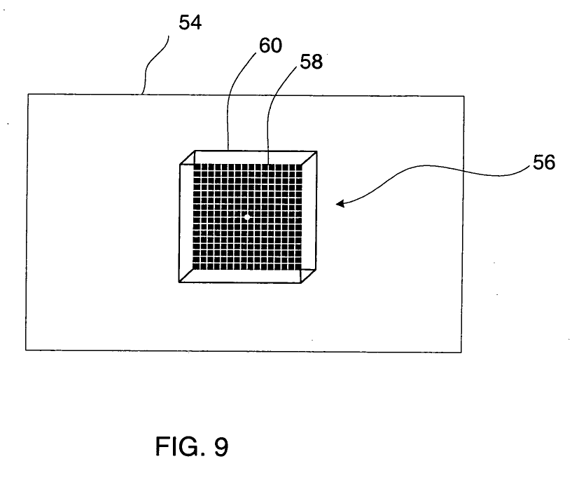

FIG. 9 is a top plane view of another exemplary viewing card 54 for use with the eye exercise device of FIGS. 1-7, having graphic indicia 56, namely an optical illusion grid pattern 58 in a “box” 60, in the form of a “magicube”. After staring at the graphic indicia over several minutes, the grid pattern will appear to flip from a “back” of the “box” to the “front” of the box, or vice-versa.

FIG. 10 is a diagrammatic view showing the exemplary embodiment of the eye exercise device 10 being used by a user U, with the contact surface 18 of the front horizontal portion 12 being placed up against the user's face and with a viewing card 50 positioned in one of the viewing card holders.

The operation of the eye exercise device 10 is now described to improve the conditions of presbyopia (farsightedness) or myopia (nearsightedness). For presbyopia, a viewing card 50F with the smallest mirror image font 50F is selected, and is placed in the viewing card holders 30A and 32B that has the shortest focal length. If the user can read this viewing card, the user does not suffer from presbyopia. However, if the user cannot clearly read the smallest mirror image font 50F (which because of the mirror is reversed back to normal text), then the user will move the viewing card one position down to establish a longer focal length (e.g. See FIG. 4.) If the user still cannot clearly read viewing card 50F, he or she will continue to move the card down until the viewing card can be read. If after moving the viewing card 50F all the way down to the lowest position 30J and 32J, the user still cannot read the viewing card 50F, he or she will select a viewing card with larger mirror image text, and repeat the process, starting at viewing card holders 50B and 52B. Each time a viewing card cannot be clearly read in the lowest position, it is replaced with a viewing card with progressively larger mirror image text. The user will eventually be able to select a viewing card and location combination that can be read comfortably. This will establish a starting point. The user will then move the selected viewing card one viewing card holder position up at a time and stare at the text until it becomes clear. The user will preferably do this at least one and preferably two or more time during the course of a day for a few minutes (e.g., about five minutes). Once the user can clearly read a viewing card, it will be moved to a higher position (to establish a shorter focal length) and repeat until the user can read the viewing card clearly. At this point, the user can repeat the process using a viewing card with yet smaller text, practice until the smaller text can be read clearly, and repeat again, until the user is working with the smallest text. From this point, the user can continue moving the viewing card with the smallest text until the uppermost position is reached. When this occurs, the user's presbyopia should be improved and reading, unaided by reading glasses should be possible. Once good close reading distance has been accomplished, the user can continue the exercise as needed to maintain the improvement. In lieu of swapping our a viewing card with larger text to progressively smaller text and eventually the smallest text at one position, and then move the viewing card with the smallest text to the uppermost position with the smallest focal length, the user can instead move a card with a certain sized text up each position until that viewing card can be clearly read at the highest position with the shortest focal length, and then repeat with viewing cards with a progressively smaller text. Regardless of the steps followed, after several weeks to months, improved close distance viewing should result.

The eye exercise device 10 can also be use to improved myopia (nearsightedness) where a person can see things near but not far. In this process, a user with myopia will select a viewing card with the smallest print (e.g., 50F), and place it in the closed position in the device and progressively move it downwardly to lengthen the focal length. If the user can clearly see the viewing card with the smallest text at the longest focal distance, then that user does not suffer from myopia. However, user's with myopia will find a focal length at which the text on the viewing card becomes unclear. The user will then take a viewing card with the next larger text and repeat. The user will stare the viewing card for several minutes (e.g. about two to five minutes) once, twice or several times a day over a period of time until the text on the viewing card can be clearly read. The process is repeated, but at a longer focal distance and progressively using viewing cards with smaller text. Once a viewer can read the viewing card with the smallest test farthest away, the user's myopia will have been improved. This may take several weeks to several months, but most users should get at least partial improvement.

Thus by using the device, there is provided a method for exercising the eyes to improve and preserve vision. One such embodiment of the method to met by:

(a) providing a device with a front end that provides a light pathway from one viewing card bearing first graphical indicia locatable at selectable focal lengths distant from the front end of the device;

(b) having a user view the one viewing card for an eye exercise period of time;

(c) changing at least one of the focal length between the one viewing card and the front end of the device and/or exchanging the one viewing card for another viewing card bearing graphic indicia of different size or sizes; and

(d) repeating step (b).

By repeating steps (a) to (d) over time, and by working with changes in the focal length and the size of the font on the viewing cards, the eye muscles can be strengthened.

The drawings in the foregoing description are not intended to represent the only form of the invention in regard to the details of its construction and manner of operation. In fact, it will be evident to one skilled in the art that modifications and variations can be made without departing from the spirit and scope of this invention. Although specific terms have been employed, they are intended in a generic and descriptive sense only and not for purposes of limitation.

I claim:

1. An eye exercise device comprising:
a first portion comprising a first end and a second end and having a longitudinal light channel formed therethrough;
a second portion having a longitudinal dimension and being positioned at an angle relative to the first portion, the first and second portions intersecting in a connection region, the second portion comprising at least one viewing card holder;
a mirror located in the connection region, the mirror being oriented to direct light along the longitudinal light channel in the first portion and the longitudinal dimension of the second portion; and
at least one viewing card adapted to be retained by the viewing card holder.

2. The eye exercise device of claim 1, wherein the second portion comprises side walls and the at least one viewing card holder comprises a plurality of holders on the side walls.

3. The eye exercise device of claim 2, wherein the plurality of holders on the side walls are spaced apart by predetermined distances and are generally parallel to each other.

4. The eye exercise device of claim 1, wherein the first end of the first portion is generally convexly curved.

5. The eye exercise device of claim 1, wherein the first portion and the second portion are formed together as a single unit.

6. The eye exercise device of claim 1, wherein the longitudinal light channel of the first portion is oriented generally perpendicularly to the longitudinal dimension of the second portion.

7. The eye exercise device of claim 1, wherein the second portion comprises a front wall, two opposite side walls, and a bottom wall and permits light to impinge on the viewing card placed in the at least one viewing card holder.

8. The eye exercise device of claim 1, further comprising a light source for illuminating the at least one viewing card.

9. The eye exercise device of claim 1, wherein the first portion and the second portion comprise opaque material.

10. The eye exercise device of claim 1, wherein the at least one viewing card bears text in mirror image.

11. The eye exercise device of claim 1, wherein a plurality of viewing card are provided, with at least two cards bearing text in mirror image of different font sizes.

12. The eye exercise device of claim 1, wherein the at least one viewing card bears graphic indicia adapted to exercise eye muscles.

13. An eye exercise device comprising:
a first portion comprising a first end and a second end and having a longitudinal light channel therethrough;
a second portion having a longitudinal dimension and positioned at an angle relative to the first portion, the first and second portions intersecting in a connection region, the second portion comprising at least one holder; and
a mirror located in the connection region that is oriented to direct light between the longitudinal channel in the first portion and the longitudinal dimension of the second portion, wherein the holder is adapted to provide a plurality of different focal lengths between the first end of the first portion, the mirror, and a position of the at least one holder

14. The eye exercise device of claim 13, further comprising at least one viewing card adapted to be retained by the at least one holder.

15. The eye exercise device of claim 13, wherein the second portion comprises a front wall, two opposite side walls, and a bottom wall.

16. The eye exercise device of claim 13, wherein the at least one viewing card bears text in mirror image.

17. The eye exercise device of claim 13, wherein a plurality of viewing card are provided, with at least two cards bears text in mirror image of different font sizes.

18. The eye exercise device of claim 13, wherein the at least one viewing card bears graphic indicia adapted to exercise eye muscles.

19. A method for exercising the eyes, comprising:
(a) providing a device with a front end that provides a light pathway from one viewing card bearing first graphical indicia locatable at selectable focal lengths distant from the front end of the device;
(b) having a user view the one viewing card for an eye exercise period of time;
(c) changing at least one of the focal length between the one viewing card and the front end of the device and/or exchanging the one viewing card for another viewing card bearing different graphic indicia; and
(d) repeating step (b).

20. The method of exercising the eyes of claim 19, wherein the graphic indicia on the one viewing card has a first size or sizes and the graphic indicia on the other viewing card has different size or sizes from that on the one viewing card.

21. The method of exercising the eyes of claim 19, wherein by repeating steps (a) to (d) over time, the eyes will be strengthened.

22. The method of claim 19, wherein the device comprises a mirror which flips the image of the graphical indicia on the viewing cards.

23. The method of claim 22, wherein the graphical indicia on the viewing cards comprises mirror image text.

24. The method of claim 19, wherein the graphical indicia on the viewing cards bear graphic indicia adapted to exercise eye muscles.
